(19) United States
(12) Patent Application Publication
(10) Pub. No.: US 2003/0113828 A1
(43) Pub. Date: Jun. 19, 2003

(54) Compositions and methods for modulating Syk function

(76) Inventors: Mark H. Ginsberg, San Diego, CA (US); Darren G. Woodside, Pearland, TX (US)

Correspondence Address: Licata & Tyrrell P.C., 66 East Main Street, Marlton, NJ, 08053, US

(21) Appl. No.: 10/293,862

(22) Filed: Nov. 12, 2002

Related U.S. Application Data
(60) Provisional application No. 60/348,220, filed on Nov. 9, 2001.

(51) Int. Cl. A61K 38/16; C12Q 1/48; C12N 9/12

(52) U.S. Cl. 435/15; 435/194; 514/13; 530/326

(57) ABSTRACT

Methods are provided for identifying agents which modulate the interaction of tyrosine kinases of the Syk family with integrins. Also provided are compositions and methods for using these agents to modulate tyrosine kinases of the Syk family and to treat disease such as thrombosis, inflammation, metastasis and tumor cell growth in a subject.

### INTRODUCTION

This application claims the benefit of priority for U.S. provisional Application Serial No. 60/348,220, filed Nov. 9, 2001, which is herein incorporated by reference in its entirety.

This invention was supported in part by funds from the U.S. government (NIH Grant No.HL 48728) and the U.S. government may therefore have certain rights in the invention.

### FIELD OF THE INVENTION

Using mimetics of integrin β3 cytoplasmic domains, it has now been found that the Syk family of non-receptor tyrosine kinases directly interact with the integrin β3 cytoplasmic tails. This interaction is unique among this family of kinases that are classically thought to be regulated by direct interaction with immune response receptors. Further, a unique site within the cytoplasmic domain has been identified that when mutated prevents the ability of integrins to bind and regulate Syk, but maintains other integrin dependent functions such as formation of focal adhesions and stress fibers, and the activation of pp125^{FAK}. The Syk family of tyrosine kinases is essential for organism viability, proper development and function of the immune system, and has been implicated in the suppression of breast cancer. The direct interaction of the Syk family of kinases with integrins provides a new therapeutic and diagnostic target for development of agents useful in the treatment and diagnosis of thrombosis, inflammation, metastasis and tumor cell growth. Agents that modulate the integrin/Syk family kinase association, particularly inhibitors, are expected to be useful in treatment of thrombosis and inflammatory diseases, and in the suppression of tumor growth. Further, since such agents prevent integrin dependent regulation of Syk only, the immuno-suppressive side effects observed upon broad inhibition of this family of kinases will be minimized.

### BACKGROUND OF THE INVENTION

Integrin adhesion receptors bind components of the extracellular matrix or cell surface molecules, and transmit signals which regulate processes such as cell proliferation, differentiation, migration and death (reviewed in Hynes, R. O. (1992) Cell 69:11-25; Schwartz et al. (1995) Ann Rev Cell Dev Biol 11:549-599). Integrin signaling is initiated by ligand binding, which is thought to involve conformational changes in integrins that are propagated to their intracellular, cytoplasmic domains. The ability of integrins to function as signaling receptors is dependent on these cytoplasmic domains, which are typically short (13-70 amino-acid residues in length) and lack known catalytic activity. Thus, integrins rely on either direct or indirect associations of their cytoplasmic domains with signaling and/or adaptor molecules to initiate signal transduction cascades.

An early event in integrin signaling in certain cells involves activation of the non-receptor tyrosine kinase Syk (Clark et al. (1994) J Biol Chem 269:28859-28864; Lin et al. (1995) J Biol Chem 270:16189-16197). In platelets, integrin αIIbβ3 engagement (Clark et al. (1994) J Biol Chem 269:28859-28864) or clustering Gao et al. (1997) EMBO J 16:6414-6425) rapidly activates Syk in a manner independent of an intact actin cytoskeleton (Lin et al. (1995) J Biol Chem 270:16189-16197; Miranti et al. (1998) Curr Biol 8:1289-1299), differentiating integrin activation of Syk from that of another tyrosine kinase, FAK. Integrins β1 (Lin et al. (1995) J Biol Chem 270:16189-16197) and β2 (Yan et al. (1997) J Immunol 158:1902-1910) have been disclosed as regulating Syk activity. Syk is essential for integrin β2-dependent morphological changes and respiratory burst in neutrophils (Fernandez, R. and Suchard, S. J. (1998) J. Immunol 160:5154-5162).

The Syk family of kinases (Syk and Zap-70) is essential for normal development and function of the immune system (Chu et al. (1998) Immunol.Rev. 165, 167-180; Turner et al. (1997) J Exp.Med. 186, 2013-2021), and Syk is required for the maintenance of vascular integrity (Turner et al. (1995) Nature 378:298-302; Cheng et al. (1995) Nature 378:303-306). These kinases are structurally distinct in that they contain tandem N-terminal SH2 domains followed by a C-terminal kinase domain. A helical “Y” shaped linker region termed “interdomain A” joins the tandem SH2 domains. Kinase activity and subcellular localization of this kinase family within immune cells can be controlled by binding of its tandem SH2 domains to a doubly phosphorylated tyrosine ligand in immune response receptors (Immunoreceptor Tyrosine-based Activation Motif, ITAM) (Chu et al. (1998) Immunol Rev 165:167-180). Linking the tandem SH2 domains with the kinase domain is the “interdomain B” region. This region contains a number of tyrosines that are phosphorylated in vivo and can recruit other signaling/adaptor molecules such as src family members (Pelosi et al. (1999) J Biol Chem 274:14229-14237), Vav-1 (Deckert et al. (1996) Immunity 5:591-604), and cbl (Meng et al. (1999) Nature 398:84-90).

### SUMMARY OF THE INVENTION

An object of the present invention is to provide a method for identifying agents which modulate the Syk family of tyrosine kinases. In this method, an agent is contacted with a protein mimetic comprising an integrin cytoplasmic domain in the presence of Syk. Any changes in the interaction of Syk with the cytoplasmic domain, which are indicative of the agent being a modulator of activity of the Syk family of tyrosine kinases, are then detected. In particular, agents which inhibit the interaction of a tyrosine kinase of the Sky family with an integrin cytoplasmic domain are desired.

Another object of the present invention is to provide compositions which modulate a tyrosine kinase of the Syk family. Compositions of the present invention comprise an agent that changes the interaction of the tyrosine kinase of the Syk family with an integrin. In a preferred embodiment, the agent inhibits the interaction of the tyrosine kinase with an integrin.

Another object of the present invention is to provide a method for modulating a tyrosine kinase of the Syk family via administration of a composition comprising an agent that changes the interaction of the tyrosine kinase of the Syk family with an integrin. In a preferred embodiment, the agent inhibits the interaction of the tyrosine kinase with an integrin.

Yet another object of the present invention is to provide a method for treating thrombosis, inflammation, metastasis or tumor cell growth in a subject by administering to the subject a composition comprising an agent which changes the interaction of the tyrosine kinase of the Syk family with an integrin. In a preferred embodiment, the agent inhibits the interaction of the tyrosine kinase with an integrin.

### DETAILED DESCRIPTION OF THE INVENTION

It has now been found that Syk and ZAP-70 can directly interact with integrin β1, β2, and β3 cytoplasmic domains with high affinity. This interaction involves the N-terminal SH2 domain of both kinases and is enhanced by the presence of the interdomain A region. Further, the Syk/ZAP-70 integrin interaction is mediated by a phosphorylation-independent interaction of an SH2 domain.

The present invention provides methods for identifying agents, compositions comprising such agents and methods of using such agents to specifically modulate, and more preferably inhibit, the interaction of tyrosine kinases of the Syk family with integrins. Such methods and agents are useful in selectively inhibiting the unwanted effects of the interaction of tyrosine kinases of the Syk family with integrins without disrupting the desired immune system functions of these tyrosine kinases. Further, Syk activation and events downstream of Syk can be blocked selectively without perturbing other signaling functions of these integrins

Syk protein tyrosine kinase is essential for immune system development and function (Chu et al. (1998) Immunol Rev 1998, 165:167-180, and for the maintenance of vascular integrity (Cheng et al. (1995) Nature 1995, 378:303-306; Turner et al.(1995) Nature 1995, 378:298-302). In leukocytes, Syk is activated by binding to di-phosphorylated immune receptor tyrosine-based activation motifs (pITAMs; Chu et al. (1998) Immunol Rev 1998, 165:167-180). Syk can also be activated by integrin adhesion receptors (Clark et al.(1994) J Biol Chem 269: 28859-28864; Gao et al.(1997) EMBO J 16:6414-6425), but the mechanism of its activation is unknown. Syk activation is an early response to integrin clustering (Miranti et al. (1998) Curr Biol 8:1289-1299) and Syk is found in a protein complex that contains integrins (Sarkar et al. (1999) Biochem J 338 (Pt 3): 677-680; Saci et al. (2000) Biochem J 351 Pt 3:669-676). Clark et al. (1994) J Biol Chem 269:28859-28864. When platelets adhered to the αIIbβ3 ligand fibrinogen, Syk co-precipitated with integrin αIIbβ3. Integrin-dependent Syk activation (Clark et al. (1994) J Biol Chem 1994, 269: 28859-28864) has been disclosed to depend on integrin cytoplasmic domains (Gao et al. (1997) EMBO J 16: 6414-6425).

The interactions between Syk and recombinant model protein mimics of dimerized integrin cytoplasmic tails were examined. The model protein mimics are designed with an N-terminal heptad repeat sequence joined to the integrin cytoplasmic domains. The repeats form a coiled-coil homodimer that dimerizes the integrin tails (Pfaff et al. (1998) J Biol Chem 273:6104-6109). These protein mimics are also describied in U.S. patent application Ser. No. 90/320,907, filed May 27, 1999 and U.S. patent application Ser. No. 90/323,447, filed Jun. 1, 1999, the teachings of which are herein incorporated by reference in their entirety. Syk and Zap-70 from cell lysates bound to a β3 tail model protein. This association was specific as there was no detectable binding to the αIIb tail or to a structure altering (Ulmer et al. (2001) Biochemistry 40:7498-7508) point mutant of the β3 tail (Y747A) that perturbs many integrin functions (Ylanne et al. (1995) J Biol Chem 270:9550-9557). Furthermore, Syk and its paralog, Zap-70, were enriched to a greater extent than talin, a protein known to interact directly with integrin β cytoplasmic tails (Pfaff et al. (1998) J Biol Chem 273: 6104-6109; Knezevic et al.(1996) J Biol Chem 271:16416-16421).

To localize β3 integrin binding sites within Syk, the binding of recombinant wild-type and mutant Syk expressed in CHO cells was examined. Wild-type Syk bound to the β3 tail. Neither kinase activity nor the kinase domain of Syk was required for binding since both a kinase-inactive Syk(K402R) and a Syk truncation mutant (residues 1-330) lacking the kinase domain bound to the β3 tail. Thus, Syk can associate specifically with the β3 integrin cytoplasmic tail, and this interaction involves the N-terminal 1-330 residues of Syk.

Recombinant fragments of Syk and Zap-70 were used to assess whether the association between integrin cytoplasmic tails and Syk family members was direct. The Syk family of non-receptor tyrosine kinases consists of N-terminal tandem SH2 domains separated by an intervening sequence termed “interdomain A” (Chu et al. (1998) Immunol Rev 165:167-180). Following the tandem SH2 domains is an “interdomain B” region and a large kinase domain (Chu et al. (1998) Immunol Rev 165:167-180). Fragments containing both SH2 and interdomains A and B [Syk(6-370) and Zap-70(1-337)] bound to the β3 tail. No binding was detected with the αIIb or β3(Y747A) tail. The tandem SH2 domains of Syk recognize phosphorylated tyrosine residues in consensus pITAM motifs [YxxI/L(x)₆₋₈YxxI/L] in immune response receptor subunits (Chu et al. (1998) Immunol Rev 165:167-180). However, integrin activation of Syk has been disclosed as being ITAM-independent (Gao et al.(1997) EMBO J 16:6414-6425). Indeed, a C-terminal SH2 domain mutant of Syk that is deficient in binding to pITAMs, but can be activated by integrin αIIbβ3 (Syk(R195A)) (Gao et al. (1997) EMBO J 16:6414-6425), bound to the β3 cytoplasmic tail. Additional mapping studies indicated that Syk(6-270) bound to the integrin β3 tail, however Syk(163-270), which contains only the C-terminal SH2 domain, failed to bind.

An R42A mutation (predicted to disrupt the phosphotyrosine binding pocket in the N-terminal SH2 domain) was introduced into the Syk(6-270) construct to test its effects on binding to the β3 tail. This mutation failed to prevent its binding to the β3 tail in direct binding assays, indicating that the Syk-β3 interaction involves a pITAM-independent mechanism.

To verify that the Syk-β3 interaction is pITAM-independent mechanism, the effect of 8000-fold molar excess of PITAM peptides on binding of Syk(6-370) to the β3 tail was examined. A dually-phosphorylated FcεRIγ ITAM peptide [DGVY(PO₃)TGLSTRNQETY(PO₃)ETLKTCR (SEQ ID NO:1)] failed to compete with the β3 integrin cytoplasmic tail for binding to Syk(6-370). pITAM peptide derived from the TCRξ chain also failed to compete. Soluble pITAM peptide was used at a concentration of 40 μM, well above the reported 2.6 nM Kd for the interaction between Syk(6-370) and the FcεRIγ pITAM peptide (Ottinger et al. (1998) J Biol Chem 273:729-735). Similar data were obtained in a quantitative enzyme-linked immunosorbent assay. The FcεRIγ pITAM peptides were functional, as they activated Syk kinase (EC₅₀ 1-2 μM) in vitro (Shiue et al.(1995) J Biol Chem 270:10498-10502). Thus, Syk recognition of β3 involves a unique specificity, distinct from Syk's interaction with pITAMs.

Deletion mutants were then used to identify regions of the β3 tail involved in Syk binding. The N-terminal deletion β3(Δ716-733) (which removes residues 716-733 of the integrin β3 cytoplasmic domain) retained the ability to bind Syk and Zap-70. However, deletion of seven more residues β3(Δ716-740) prevented binding. Deletion of the last 4 C-terminal (Tyr⁷⁵⁹-Arg-Gly-Thr⁷⁶²) residues [β3(759X)] abolished Syk interaction with β3 as did removal of 11 C-terminal residues [residues Thr⁷⁵²-Thr⁷⁶², (β3752X)]. Thus, β3 residues Arg⁷³⁴-Thr⁷⁶² are sufficient for its direct interaction with Syk. Furthermore, removal of the 4 C-terminal residues of integrin β3 abrogates binding in vitro.

The effect of deletion of these residues on the association between Syk and integrin αIIbβ3 was also assessed. CHO cells expressing αIIbβ3(759X) were generated. These cells expressed similar quantities of αIIbβ3 and adhered normally to fibrinogen, but unlike wild type αIIbβ3, αIIbβ3(759X) was not co-immunoprecipitated with Syk from these adherent cells. When integrin αIIbβ3(759X)-expressing CHO cells were plated on fibrinogen, they spread and formed focal adhesions, but did not support adhesion-dependent Syk phosphorylation. In contrast, these cells exhibited adhesion-dependent phosphorylation of another tyrosine kinase, pp125^{FAK}. Thus, the physical association of integrin αIIbβ3 with Syk requires the Syk binding function of the β3 cytoplasmic tail, and is required for integrin-dependent activation of Syk. These results also indicate that distinct β3 cytoplasmic domain structural features are responsible for activation of Syk and pp125^{FAK}.

Syk activation upon integrin engagement is rapid, and unlike pp125^{FAK} activation, is insensitive to actin depolymerizing agents (Miranti et al. (1998) Curr Biol 8:1289-1299). The tandem SH2 domains of Syk were used as a dominant-negative inhibitor to further examine the dichotomy between integrin-dependent regulation of Syk and pp125^{FAK}. Low levels of Syk(1-330) expression did not lead to detectable inhibition of integrin-dependent Syk activation (Gao et al. (1997) EMBO J 1997 16:6414-6425). However, overexpression of Syk(1-330) in CHO cells stably expressing αIIbβ3 and a single genetic copy of Syk blocked the integrin-dependent phosphorylation of Syk. Vav1, a Rac guanine nucleotide exchange protein, is phosphorylated and activated upon binding to phosphorylated Tyr³⁴⁸ of Syk (Chu et al. (1998) Immunol Rev 165:167-180). Over expression of Syk(1-330) also inhibited adhesion dependent phosphorylation of Vav1. These results confirm that sequences within the N-terminal half of Syk are involved in its activation via binding to the β3 tail. Overexpression of Syk(1-330) did not, however, inhibit adhesion-induced phosphorylation of pp125^{FAK}. Thus, Syk activation requires a distinct integrin-dependent signaling pathway from that which activates pp125^{FAK}, and Syk activation and events downstream of Syk can be blocked selectively without perturbing certain other signaling functions of β3 integrins.

Syk and Vav1 cooperate to remodel the actin cytoskeleton by inducing Rac-dependent lamellipodia formation (Miranti et al. (1998) Curr Biol 8:1289-1299). To assess the functional effects of the integrin-Syk interaction, CHO cells stably expressing wild-type αIIbβ3 or αIIbβ3(759X) were co-transfected with Vav1, or Syk and Vav1, and integrin-dependent lamellipodia formation was assessed. Fibrinogen-adherent cells expressing integrin αIIbβ3(759X) generated at least one actin-rich lamellipodia in 28.3±1.0 percent of cells transfected with Vav1. Co-transfection of Syk and Vav1 did not increase this response (26.3±1.9 percent). In sharp contrast, transfection of Syk and Vav1 into cells expressing wild-type αIIbβ3 resulted in a dramatic increase in the extent of lamellipodia formation (from 24.0±2.2 in Vav1 transfected cells to 53.3±5.6 percent in Syk/Vav1 transfected cells). Thus, the interaction between Syk and the β3 integrin cytoplasmic domain initiates Syk-dependent cytoskeletal re-organization.

These results are indicative of a novel paradigm for the regulation of Syk kinases. The mechanism of Syk activation by integrins differs from that of immune receptors. pITAMs within immune receptors serve as the binding sites which recruit Syk through its tandem SH2 domains. In contrast, neither the phosphotyrosine binding sites within the Syk SH2 domains, nor phosphorylation of tyrosines in the β3 tail are required for Syk interaction with, or activation (Gao et al. (1997) EMBO J 16:6414-6425) by, β3 integrins. Thus, integrins and immune receptors have evolved distinct mechanisms for recruitment of Syk to transmembrane receptor complexes. Syk is recruited to clustered integrins by its direct interaction with integrin cytoplasmic domains. Src kinases are present in these integrin-dependent protein complexes (Hruska et al. (1995) Endocrinology 136:″2984-2992) and are required for maximal integrin-dependent activation of Syk (Gao et al. (1997) EMBO J 16:6414-6425). Thus, the proximity promoted by integrin clustering may promote Syk trans-phosphorylation by one or more Src family kinases(Chu et al. (1998) Immunol Rev 165:167-180) leading to activation of Syk catalytic activity (El Hillal et al. (1997) Proc Natl Acad Sci USA 94:1919-1924).

The dependency of the interaction of integrins with Syk and ZAP-70 on the kinases' N-terminal SH2 domain and inter-domain A region was demonstrated in direct binding assays. In these experiment, the interaction between Syk and Zap-70 N-terminal SH2 domains generally appeared to be of lower affinity than the tandem SH2 domains together. Also, removal of the N-terminal SH2 domain of Syk decreased, but did not prevent, binding of Syk to the integrin β3 cytoplasmic domain. These results are indicative of regions in addition to the N-terminal SH2 domains of Syk and Zap-70 also be involved in binding to the integrin β3 tail. Because the C-terminal SH2 domain of Syk and Zap-70 failed to bind the integrin β3 cytoplasmic domain, the interdomain A region was examined further. The interdomain A of Syk was expressed as a GST-fusion protein to determine if it could directly interact with the cytoplasmic domain of β3. No binding was detected. The orientation of the SH2 domains of Syk and Zap-70 are such that the phosphotyrosine binding domains bind to dually phosphorylated ITAM sequences (YxxI/Lx(6-8)YxxI/L) in a reverse colinear fashion (Futterer et al. (1998) J Mol Biol 281:523-537; Hatada et al. (1995) Nature 377:32-38). The tandem SH2 domains of SHP-2 are oriented differently from those of Syk and Zap-70. The regions involved in phosphotyrosine binding are widely separated and in opposite orientation (Hof et al. (1998) Cell 92:441-450). When tested in a direct binding assay, the tandem SH2 domains of SHP-2 did not bind the β3 tails. Thus, interaction with the integrin β3 cytoplasmic tail is not a general property of tandem SH2 domain containing proteins. When the Syk interdomain A was inserted into the interdomain region of SHP-2, SHP-2 now bound to the integrin β3 cytoplasmic domain.

To examine the role of the interdomain A region of Zap-70, a series of truncation mutants were tested for binding to the integrin β3 cytoplasmic domain. The N-terminal SH2 domain of Zap-70, when expressed in conjunction with its intact IA domain (Zap-70(1-162)), bound β3 to a similar extent as the tandem SH2 domains. However, removal of the C-terminal half of interdomain A (residues Leu¹³³-Pro¹⁶², Zap-70(1-132)) resulted in a decrease in binding similar to levels of the N-terminal SH2 domain alone. Thus, the interdomain A is necessary for optimal binding of tyrosine kinases of the Syk family such as Syk and Zap-70 to the β3 integrin cytoplasmic domain and confers binding to the SHP-2 tandem SH2 domains. This region of Zap-70 forms a helix-turn-helix motif in very close proximity to the N-terminal SH2 domain (Hatada et al. (1995) Nature 377:32-38). Interestingly, the corresponding sequence in Syk (Leu¹³⁸-Pro¹⁶⁷) has a similar structure (Futterer et al. (1998) J Mol Biol 281:523-537), and shares 97% sequence similarity and 83% amino acid identity.

As demonstrated herein, the binding of Syk to β3 involves the N-terminal SH2 domain and does not require phosphorylation of the two tyrosines contained in the β3 tail. However, mutation of these two tyrosine residues to phenylalanine (β3(Y747,759F)) within the β3 cytoplasmic domain can alter αIIbβ3 dependent functions (Law wt al. (1999) Nature 401:808-811). Therefore the effect of the β3(Y747,759F) mutation on Syk or Zap-70 binding to the β3 tail was tested. The β3(Y747,759F) mutations had no effect on the binding of Syk or Zap-70 to the β3 cytoplasmic domain.

Phosphorylation of the β3 integrin cytoplasmic domain occurs as a consequence of integrin engagement (Law et al. (1996) J Biol Chem 271:10811-10815). To determine if phosphorylation of the β3 cytoplasmic domain could affect Syk binding, competition assays were performed using tyrosine phosphorylated or non-phosphorylated peptides in the context of the last 23 amino acids of the β3 cytoplasmic domain. Peptides were used at a concentration 1×10⁻⁴ M, and Syk was used at a concentration of 5×10⁻⁹ M. The non-phosphorylated peptide competed for integrin β3 tail binding to Syk. In three experiments, there was a 52±3.4% decrease in Syk binding to the β3 tail model proteins in the presence of non-phosphorylated β3-23 peptide as compared to the phosphorylated peptide (β3-23P). The phosphorylated peptide did not detectably compete, although this peptide preparation is active in binding Shc (Cowan et al. (2000) J Biol Chem 275:36423-36429). Thus, substitution of Phe residues at β3 Tyr^{747,759} does not disrupt Syk binding, confirming the lack of requirement for phosphorylation. Furthermore, phosphorylation of β3(Y747,759) reduces its capacity to compete for Syk binding to the unphosphorylated β3 tail.

Mice expressing mutant integrin αIIbβ3(Y747,759F) manifest mild bleeding accompanied by unstable platelet aggregates and reduced clot retraction (Law et al. (1999) Nature 401:808-811). These defects have been ascribed to inhibition of binding of p-Tyr dependent ligands such as myosin (Jenkins et al. (1998) J Biol Chem 273:13878-13885) or Shc (Cowan et al. (2000) J Biol Chem 275:36423-36429) to the β3 tail (Phillips et al. (2001) Curr Opin Cell Biol 13:546-554). Results shown herein are indicative of the alternative possibility that interrupting tyrosine phosphorylation of the β3 cytoplasmic domain may perturb platelet function by prolonging association of Syk with the β3 tail, leading to prolonged signaling. Syk signaling is known to result in elevated cytoplasmic Ca⁺⁺ which could promote calpain dependent cleavage of cytoskeletal proteins and thus block clot retraction. Further analysis of the signaling properties of these mutant platelets and the effect of combining this mutation with Syk deficiency will permit an evaluation of this potential mechanism of platelet dysfunction.

The tandem SH2 domains of these kinases bind to multiple integrin β cytoplasmic domains with varying affinities (β3(Kd=24 nM)>β2 (Kd=36 nM)>β1(Kd=76 nM) as judged by both affinity chromatography and surface plasmon resonance. Thus, these integrin cytoplasmic domains directly bind Syk with relatively high affinity and this interaction is likely to account for integrin β1, β2, and β3 regulation of Syk function. As measured by surface plasmon resonance, the affinity of the integrin β3 cytoplasmic domain for Syk was 24×10⁻⁹ nM in contrast to the tenfold higher affinity of Syk for the dually phosphorylated FcεRIγ-ITAM, 2.6×10⁻⁹ M (Ottinger et al. (1998) J Biol Chem 273:729-735). However integrins are far more abundant than ITAM containing receptor complexes on most cells. For example, Jurkat T cells express ˜80,000 copies of α4β1 (Chen et al. (1999) J Biol Chem 274:13167-13175) and only ˜-12,000 copies of the TCR (Graber et al. (1991) J Immunol 146:2935-2943) on their cell surface. Syk colocalizes with αIIbβ3 integrins in lamellipodia. Integrin dependent recruitment of this kinase family to lamellipodia is believed to contribute to the mechanism whereby polarized migrating lymphocytes are more sensitive to antigenic stimulation at their leading edge. pITAM binding to Syk directly regulates its functions. Thus, the interaction with integrin cytoplasmic domains, and in particular the interaction with β3 integrin shown herein, is believed to serve to modulate or focus the regulation of Syk and ZAP-70 by immune response receptors.

Accordingly, the present invention provides methods for identifying agents that modulate the Syk family of tyrosine kinases through modulation of their interaction with an integrin cytoplasmic domain. In these methods, a protein mimetic comprising an integrin cytoplasmic domain such as described herein is contacted with the agent in the presence of a tyrosine kinase of the Syk family. In a preferred embodiment, the protein mimetic comprises a β3 integrin cytoplasmic domain. Changes in the interaction of tyrosine kinase of the Syk family with the integrin cytoplasmic domain of the protein mimetic in the presence of the agent are then detected. Any changes in the interaction of the tyrosine kinase with the integrin cytoplasmic domain are indicative of the agent being a modulator of activity of the Syk family of tyrosine kinases. In a preferred embodiment, the agent inhibits the interaction of the tyrosine kinase with the integrin cytoplasmic domain.

The present invention also provides compositions which modulate, or more preferably inhibit the interaction of a tyrosine kinase of the Syk family with an integrin cytoplasmic domain. Composition of the present invention comprise an agent which changes, or more preferably inhibits, the interaction of the tyrosine kinase of the Syk family with an integrin. In a preferred embodiment, the composition comprises an agent that changes the direct interaction of the tyrosine kinase of the Syk family with integrin β3. Exemplary agents demonstrated herein to inhibit the interaction of a tyrosine kinase of the Syk family with an integrin include the tandem SH2 domains of Syk and a peptide comprising the comprising 23 amino acids of an end terminus of a β3 cytoplasmic domain. However, additional agents can be identified routinely by those of skill in the art in accordance with the teachings provided herein. In a preferred embodiment, the agent is identified via detecting modulation of the interaction of a Syk family kinase with a protein mimetic comprising an integrin cytoplasmic domain in the presence of the agent. Such compositions can then be used to treat thrombosis, inflammation, metastasis or tumor cell growth in a subject.

The following nonlimiting examples are provided to further illustrate the present invention.

### EXAMPLES

### Example 1

### Cells, cDNAs, Peptides

The CHO cell line expressing αIIbβ3(759X) was generated by transfection of CHO cells with αIIb and β3(759X) cDNA. The CHO A5 cell line stably expressing Syk-HA was created by retroviral transduction with a C-terminal HA-tagged variant of Syk (pLHCX Syk-HA), followed by selection in hygromycin. The CHO cell line A5 stably expressing integrin αIIbβ3 as described by Hughes et al. ((1996) J Biol Chem 271:6571-6574) and was maintained in DMEM medium supplemented with 10% FCS, L-glutamine, penicillin/streptomycin, and 0.5 mg/ml G418 (Gibco-BRL). Cells were grown at 37° C. in 6% CO₂.

Mammalian Syk expression vectors have been described (Gao et al. (1997) EMBO J 16:6414-6425), along with the bacterial expression vector GST-Syk(6-370)(Shiue et al. (1995) Mol Cell Biol 15:272-281). A cDNA encoding residues 1-337 of human Zap-70 was cloned into pGEX-2T (Amersham Pharmacia Biotech).

Phospo-ITAM's consisted of the dually-phosphorylated FcεRIγ chain ITAM-DGVY(PO₃)TGLSTRNQETY(PO₃)ETLKTCR (SEQ ID NO:1) and the dually-phosphorylated TCRξ chain ITAM-NQLY(PO₃)NELNLGRREEY(PO₃)DVLV (SEQ ID NO:2) (Shiue et al. (1995) J Biol Chem 270:10498).

Anti-Syk mAb 4D10, anti-GST mAb, and polyclonal anti-His were purchased from Santa Cruz Biotechnology (Santa Cruz, Calif.). HRP-conjugated secondary goat anti-mouse and goat anti-rabbit F(ab′)₂ were purchased from Biosource International (Camarillo, Calif.).

Expression vectors encoding GST-Syk(6-370), GST-Syk(6-270), GST-Syk(163-270) have been described (Shiue et al. (1995) Mol Cell Biol 15:272-281). GST-Syk(6-108) was produced by inserting a stop codon at residue 109 of Syk by Quikchange™ (Stratagene) site-directed mutagenesis. Construction of pGEX/GST-Syk(1-370,Δ6-109) and the expression vector EMCV/myc-syk Δ(6-109) was performed by first introducing a blunt-end StuI site at position 109 in both vectors with the QuikChange system. pGEX/GST-Syk(6-370) was cut with StuI and NheI to remove the N-terminal SH2 domain of Syk. Gel purified vector was ligated to the annealed oligos 5′-gatccgccagcagcggc (SEQ ID NO:3) and 3′-gcggtcgtcgccg (SEQ ID NO: 4) encoding amino acid residues 2-5 of Syk. For EMCV/myc-Syk, vector was digested with StuI and BamHI, gel purified, and ligated with the annealed oligos 5′-ctagcagcggc (SEQ ID NO:5) and 3′-gtcgccg (SEQ ID NO:6) encoding amino acids 2-5 of Syk. All site directed mutagenesis and deletion mutations were verified by sequence analysis. GST-Zap-70(1-337), GST-Zap-70(1-103), and GST-Zap-70(163-254) were generated by PCR amplification of wild type Zap70 cDNA (pBS-Zap-70) and cloned into pGEX-2T.

### Example 2

### Affinity Chromatography/direct Binding Assays

Model protein mimics of integrin cytoplasmic domains αIIb, β1A, and β3 were prepared in accordance with procedures described by Pfaff et al. (1998) J Biol Chem 273:6104-6109). The β2 integrin cytoplasmic domain model protein was developed by PCR amplification of β2 cDNA using 5′-ccaagcttctgatccacctgagcgacctccgg (SEQ ID NO:7) and 3′-ttggggttcaaacgactctcaatcatccctagggg (SEQ ID NO:8) primers. This primer set introduced a 5′ HindIII restriction site into the N-terminal region of the β2 cytoplasmic domain, resulting in the mutation, A725L. The 3′ primer contained a BamHI restriction site immediately downstream of dual stop codons. The PCR product was cloned into pCR2.1 as described in the TA Cloning® system (Invitrogen, Carlsbad, Calif.). After sequencing, pCR2.1-β2cyt was digested with BamHI and HindIII, and subcloned into a modified bacterial expression plasmid pET15bm (lacking the HindIII restriction site). All model proteins were verified by sequence analysis, expressed, HPLC purified and verified by electrospray ionization mass spectroscopy as described by Pfaff et al. ((1998) J Biol Chem 273:6104-6109).

For affinity chromatography, Ni⁺⁺-beads coated with integrin cytoplasmic tails (5 μl packed) were added to 0.5 mg clarified cell lysate (affinity chromatography) or GST-fusion protein (5 nM) in a final volume of 0.5 ml binding buffer. Following incubation, beads were washed and bound protein was analyzed by immunoblotting. For pITAM peptide competition assays, GST-Syk(6-370) was pre-incubated with 40 μM pITAMs, then direct binding assays were performed.

For direct binding assays, model peptides (1 mg) were dissolved in 1 ml buffer containing 20 mM Tris (pH 7.9), 500 mM NaCl, and 6 M urea. To this, 50 ul packed His-Bind® Resin (Novagen, Madison, Wis.) was added and the mixture was rotated for 1 hour at room temperature. Immobilized peptides were washed 5× in above buffer without urea, and resuspended in 500 μl buffer containing 20 mM PIPES pH 6.8, 50 mM NaCl, 3 mM MgCl₂, 150 mM sucrose, 50 mM NaF, 40 mM Na₄P₂O₇*10H₂O (buffer A) supplemented with 0.1% TritonX-100 and 3 mM MgCl₂. Purified GST-fusion proteins were added to 0.5 ml buffer A supplemented with 0.1% TritonX-100 and 3 mM MgCl₂. Five μl of total packed resin loaded with various integrin cytoplasmic domain model proteins was added to the GST-fusion protein. The mixture was incubated for 40 minutes at room temperature with continuous rotation. The beads were then washed five times in buffer A with 0.1% TritonX-100 and 3 mM MgCl₂. Bound proteins were eluted by boiling in reducing sample buffer, fractionated by SDS PAGE, and immuno-blotted.

For peptide competition assays, peptides were pre-incubated with Syk(6-370) for one hour before direct binding assays were performed. Peptides were used at a concentration of 1×10⁻⁴ M, and Syk(6-370) was used at a concentration of 2×10⁻⁹ M. Bound Syk was detected as described above. The concentration of integrin cytoplasmic tail model protein estimated in the direct binding assay is 1.7×10⁻⁵ M.

### Example 3

### Elisa Competition Assay

GST-Syk(6-370) was immobilized onto Immobilon II Elisa plates (Corning) in 0.1 M NaHCO₃ (pH 8.0) for 2 hours at room temperature. After blocking with heat-inactivated BSA, plates were washed and phospho-peptide FcεRIγ ITAM was incubated at 10 μM. Without washing, soluble His-tagged integrin cytoplasmic domain model protein was added at a final concentration of 2.5 μM. After a 1 hour incubation bound integrin cytoplasmic domain model proteins were detected with anti-His antibodies and secondary HRP-conjugated antibodies.

### Example 4

### Phosphorylation and Coprecipitation

Adhesion-dependent phosphorylation assays were carried out as performed in accordance with procedures described by Gao et al. ((1997) EMBO J 16:6414-6425). For co-precipitation, cells (CHO cells or platelets) on BSA or fibrinogen (1 hour at 37° C.) were lysed in 50 mM Tris (pH 7.4) containing 0.5% Nonidet P-40, 50 mM NaCl, and a protease inhibitor cocktail (Boehringer Mannheim, Germany). After clarification at 12,000 rpm for 20 minutes, 500-750 μg of lysate were incubated with primary antibody overnight at 4° C. (polyclonal antibody 8053 for β3 integrins, or polyclonal antibody 0134 for Syk). Protein-A beads (25 μl packed, Amersham Pharmacia Biotech ) were then added and rotated for 2 hours at 4° C.

### Example 5

### Confocal Microscopy

Lamellipodia quantification was performed in accordance with procedures described by Miranti et al. ((1998) Curr Biol 8:1289-1299).

### Example 6

### Purification of GST-fusion Proteins

GST fusion proteins were produced in accordance with procedures described by Shiue et al. ((1995) Mol Cell Biol 15:272-281). Briefly, exponential growth phase bacteria were induced with 0.1 mM IPTG for 4 hours at 37° C. Cells were then pelleted, resuspended in lysis buffer (PBS with 0.5% TritonX-100, 1 mM DTT, 1 mM PMSF, and protease inhibitors (Boehringer Mannheim, Germany)), and sonicated at 4° C. Lysates were clarified at 20,000×g for 30 minutes, and supernatant was incubated with Glutathione Sepharose™ 4B (Amersham Pharmacia Biotech, Piscataway, N.J.) pre-equilibrated in lysis buffer. The mixture was incubated overnight at 4° C., then washed 4× in elution buffer (100 mM Tris pH 8.0, 100 mM NaCl, 1 mM DTT, 1 mM PMSF) without GSH. Final elution was carried out in elution buffer containing 20 mM Glutathione. For BIAcore analysis of Syk binding to integrin cytoplasmic domains, Syk(6-370) was cleaved from GST-Syk(6-370) coated Glutathione Sepharose™ 4B by thrombin (1 Unit/mg protein) overnight at 4° C.

### Example 7

### Surface Plasmon Resonance

BIAcore 3000 (BIAcore, Uppsala, Sweden) was used for real time kinetic analysis of the Syk/integrin cytoplasmic domain interactions. Experimental procedures, integrin cytoplasmic domain modifications, and data analysis was performed in accordance with procedures described by Yan et al. ((2001) J Biol Chem 276:28164-28170).

What is claimed is:

1. A method for identifying agents which modulate the Syk family of tyrosine kinases comprising:
contacting an agent with a protein mimetic comprising an integrin cytoplasmic domain in the presence of Syk; and
detecting changes in the interaction of Syk with the cytoplasmic domain, wherein any changes in the interaction of Syk with the cytoplasmic domain are indicative of the agent being a modulator of activity of the Syk family of tyrosine kinases.

2. The method of claim 1 wherein the cytoplasmic domain comprises a β3 cytoplasmic domain.

3. A composition which modulates a tyrosine kinase of the Syk family comprising an agent which changes the interaction of the tyrosine kinase of the Syk family with an integrin.

4. The composition of claim 3 wherein the agent changes direct interaction of the tyrosine kinase of the Syk family with integrin β3.

5. The composition of claim 4 wherein the agent is a peptide comprising 23 amino acids of an end terminus of a β3 cytoplasmic domain.

6. A composition which modulates a tyrosine kinase of the Syk family comprising an agent identified in accordance with the method of claim 1 which changes the direct interaction of the tyrosine kinase of the Syk family with an integrin.

7. A method for modulating a tyrosine kinase of the Syk family comprising administering a composition of claim 3.

8. A method for treating thrombosis, inflammation, metastasis or tumor cell growth in a subject comprising administering to the subject a composition of claim 3.


SEQUENCE LISTING (8 sequences)

SEQ ID NO: 1 - protein, 22 aa, Artificial sequence
  Synthetic
DGVYTGLSTRNQETYETLKTCR

SEQ ID NO: 2 - protein, 19 aa, Artificial sequence
  Synthetic
NQLYNELNLGRREEYDVLV

SEQ ID NO: 3 - DNA, 17 bp, Artificial sequence
  Synthetic
gatccgccagcagcggc

SEQ ID NO: 4 - DNA, 13 bp, Artificial sequence
  Synthetic
gcggtcgtcgccg

SEQ ID NO: 5 - DNA, 11 bp, Artificial sequence
  Synthetic
ctagcagcggc

SEQ ID NO: 6 - DNA, 7 bp, Artificial sequence
  Synthetic
gtcgccg

SEQ ID NO: 7 - DNA, 32 bp, Artificial sequence
  Synthetic
ccaagcttctgatccacctgagcgacctccgg

SEQ ID NO: 8 - DNA, 35 bp, Artificial sequence
  Synthetic
ttggggttcaaacgactctcaatcatccctagggg